(19) United States
(12) Patent Application Publication
(10) Pub. No.: US 2026/0026682 A1
(43) Pub. Date: Jan. 29, 2026

(54) SHIELDED ENDOILLUMINATION

(71) Applicant: Alcon Inc., Fribourg (CH)

(72) Inventor: Michael Sam Cardamone, Keller, TX (US)

(21) Appl. No.: 19/256,614

(22) Filed: Jul. 1, 2025

Related U.S. Application Data
(60) Provisional application No. 63/674,443, filed on Jul. 23, 2024.

(51) Int. Cl. A61B 1/06

(52) U.S. Cl. CPC A61B 1/0676; A61B 1/0607; A61B 1/0627

(57) ABSTRACT

An apparatus for surgery on an eye. The apparatus may include a cannula having a hub with an instrument port, a shield, and a sleeve. The sleeve may have a first end coupled to the instrument port and a second end coupled to the shield. The system may further comprise an illuminator having a first end configured to be coupled to a light source and a second end configured to be inserted through the instrument port and the sleeve. The shield can be configured to partially block light emitted from the second end of the illuminator to prevent glare.

### TECHNICAL FIELD

The disclosure relates generally to surgical endoillumination, including, without limitation, endoillumination during eye surgery.

### BACKGROUND

The human eye can suffer a variety of maladies causing mild deterioration to complete loss of vision. While contact lenses and eyeglasses can compensate for some ailments, ophthalmic surgery may be required for others. For example, retinal detachment, traction retinal detachment, and trauma remain major causes of visual loss worldwide, despite continuing advances in vitreoretinal care, and pars plana vitrectomy is a leading management modality for the treatment for such conditions.

While the benefits of ophthalmic surgical procedures are known, improvements to surgical systems, components, and processes can continue to improve outcomes and benefit patients. For example, adequate visualization of interior portions of the eye is important to the success of ophthalmic surgery, and improvements to visualization can greatly enhance success of the procedures.

### BRIEF SUMMARY

New and useful systems, apparatuses, and methods for surgical endoillumination are set forth in the appended claims. Illustrative embodiments are also provided to enable a person skilled in the art to make and use the claimed subject matter.

For example, some embodiments may comprise a shielded cannula for chandelier endoillumination. In some embodiments, a cannula may have a pointed shield, which can be rotated to reduce or eliminate glare. The shape of the shield can be configured to cover the end of a light fiber to stop illumination from coming directly back up the visual axis of a surgical microscope.

More generally, some embodiments relate to a system for surgery on an eye, and the system may include a cannula having a hub with an instrument port, a shield, and a sleeve. The sleeve may have a first end coupled to the instrument port and a second end coupled to the shield. The system may further comprise an illuminator having a first end configured to be coupled to a light source and a second end configured to be inserted through the instrument port and the sleeve. The shield can be configured to partially block light emitted from the second end of the illuminator.

In some embodiments, the shield may be characterized as a truncated hollow cylinder. In other embodiments, the shield may be characterized as a half cylinder.

In some embodiments, the shield and the sleeve may be coaxial. Additionally, or alternatively, the shield may have a first outside radius, and the sleeve may have a second outside radius equal to the first outside radius.

In more specific embodiments, the shield may include a base coupled to the sleeve and a face that is tilted at an angle to the base. Additionally, or alternatively, in some embodiments the base may be characterized by a center and an arc that subtends an angle having a vertex at the center. In some embodiments, it may be advantageous for the angle between the face and the base to be less than or equal to 90 degrees, and for the angle centered at the vertex to be less than or equal to 180 degrees.

Additionally, or alternatively, the shield may be configured to be rotated around the illuminator in some embodiments.

In other embodiments, a system for surgery on an eye may comprise a light source, a cannula, and an illuminator. The light source may be configured to provide an incoherent light. The cannula may have a hub with an instrument port, a shield, and a sleeve having a first end coupled to the instrument port and a second end coupled to the shield. The illuminator may have a first end configured to be coupled to the light source and a second end configured to be inserted through the instrument port and the sleeve. The sleeve and the shield can be configured to be inserted into the eye, and the shield can be configured to partially block light emitted from the second end of the illuminator in the eye.

In more specific embodiments, the shield and the sleeve may be coaxial. Additionally, or alternatively, the shield may have a first outside radius, and the sleeve may have a second outside radius equal to the first outside radius. In some aspects, the shield may include a base coupled to the sleeve, and a face that is tilted at an angle to the base. The face may intersect the base in some embodiments. Some embodiments of the base may be characterized by a center and an arc that subtends an angle having a vertex at the center. In some embodiments, it may be advantageous for the angle between the face and the base to be less than or equal to 90 degrees, and for the angle centered at the vertex to be less than or equal to 180 degrees. Additionally or alternatively, some embodiments of the shield may be configured to be rotated around the illuminator.

Some embodiments may relate to an apparatus for eye surgery. The apparatus may include a light source configured to provide an incoherent light; a cannula having a hub with an instrument port, a sleeve, and a shield; and an illuminator configured to be coupled to the light source. The sleeve and the shield may be coaxial in some embodiments. The shield may have a first outside radius, and the sleeve may have a second outside radius equal to the first outside radius. The shield may include a base coupled to the sleeve, and a face that may intersect the base at an angle that is less than or equal to 90 degrees. The base may be characterized by a center and an arc that subtends a central angle that is less than or equal to 180 degrees. The sleeve and the shield can be configured to be inserted into an eye, and the illuminator can be configured to be inserted through the instrument port and the sleeve. The shield can be configured to be rotated around the illuminator and to partially block light emitted from the illuminator into the eye.

Features, elements, and aspects described in the context of some embodiments may also be omitted, combined, or replaced by alternative features. Other features, objectives, and advantages are described in greater detail below with reference to the accompanying drawings of illustrative embodiments.

### BRIEF DESCRIPTION OF THE DRAWINGS

The accompanying drawings illustrate some features, objectives, and advantages of some embodiments of the claimed subject matter. Like reference numbers represent like parts in the examples.

FIG. 1 is a functional block diagram of an example of a system for surgical endoillumination.

FIG. 2 is a perspective view of an example of a cannula that may be associated with some embodiments of the system of FIG. 1.

FIG. 3 is another perspective view of the cannula of FIG. 2, illustrating additional details that may be associated with some embodiments.

FIG. 4 is a detail view of FIG. 3, illustrating additional details of a glare shield that may be associated with the cannula of FIG. 2.

FIG. 5 is a perspective view of another example of the glare shield.

FIG. 6 is a perspective view of another example of the glare shield.

FIG. 7 is a schematic diagram of a method for using the system of FIG. 1 with an eye.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position for ophthalmic surgery. However, as should be recognized by those skilled in the art, this frame of reference is merely a descriptive expedient rather than a strict requirement.

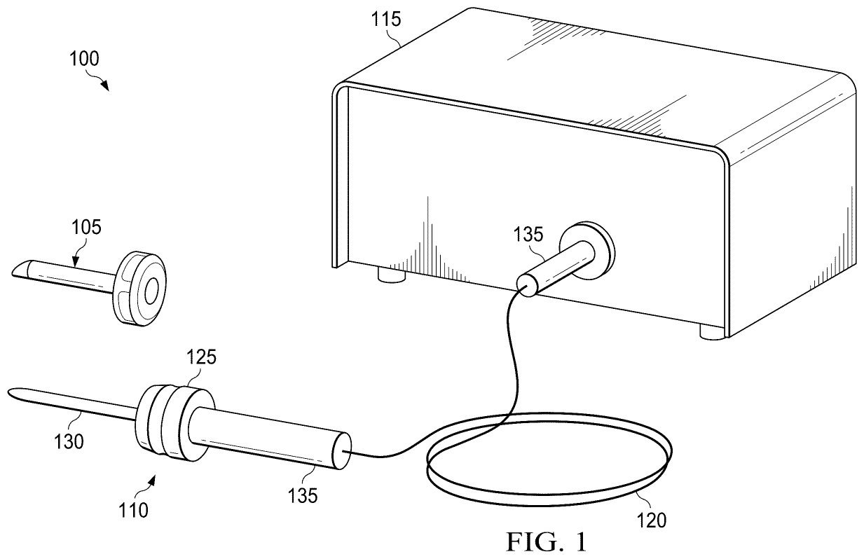

FIG. 1 is a functional block diagram of an example of a system 100 for surgical endoillumination. As shown in the example of FIG. 1, some embodiments of the system 100 may comprise a cannula 105, an illuminator 110, and a light source 115. The illuminator 110 may have a first end configured to be coupled to the light source 115 and a second end configured to be coupled to the cannula 105. For example, a cable 120 can couple the illuminator 110 to the light source 115, and the illuminator 110 may comprise a handpiece 125 and a stem 130, which can be configured to be inserted through the cannula 105.

In some examples, the light source 115 can be a xenon light source, a halogen light source, or any other light source capable of delivering incoherent light through the cable 120. The cable 120 may be an optical cable, such as an optical fiber, and may have a coupler 135 at each end to facilitate coupling the cable 120 to the illuminator 110 and the light source 115.

In general, components of the system 100 may be coupled directly or indirectly. For example, the cannula 105 may be directly coupled to the illuminator 110 and may be indirectly coupled to the light source 115 through the illuminator 110. Components may also be coupled by virtue of physical proximity, being integral to a single structure, or being formed from the same piece of material. Coupling may include optical, fluid, mechanical, thermal, electrical, or chemical coupling (such as a chemical bond), or some combination of coupling in some contexts. For example, the cannula 105 may be mechanically coupled to the illuminator 110, and the illuminator may be mechanically and optically coupled to the light source 115. Components may also include or comprise interfaces or ports to facilitate coupling and de-coupling other components, such as the coupler 135.

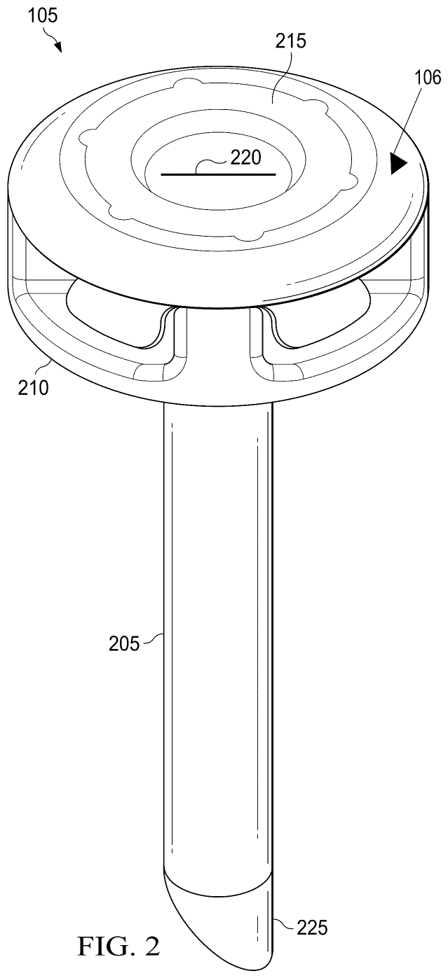

FIG. 2 is a perspective view of an example of the cannula 105 that may be associated with some embodiments of the system 100. For example, the cannula 105 may be configured for insertion into an eye to facilitate insertion and removal of instruments, such as the illuminator 110. Some examples of the cannula 105 may include a sleeve 205 and a hub 210. In some examples, a seal 215 may be coupled to the hub 210. For example, the seal 215 may be disposed at least partially over the hub 210 to form an over-molded seal. In some embodiments, the cannula 105 may comprise an instrument port 220. For example, the instrument port 220 may be a slit valve disposed at one end of the cannula 105, such as in an exposed surface of the seal 215, which may be normally closed and configured to be opened by an instrument inserted into the cannula 105. In the absence of a surgical instrument, the seal 215 and the instrument port 220 may inhibit fluid flow through the seal 215. The hub 210 may be coupled to a first end of the sleeve 205, and a glare shield 225 may be coupled to a second end of the sleeve 205. In some embodiments, an indicator 106 (e.g., an arrow or triangle shape) may be provided on cannula 105 to indicate an orientation of the glare shield (e.g., may be aligned with an apex of the glare shield as shown in FIG. 2).

The seal 215 may be made of an elastomer, such as silicone. In some embodiments, the seal 215 may be attached to the hub 210 to inhibit rotation of the seal 215 relative to the hub 210. In some embodiments, a friction fit may secure the seal 215 to the hub 210. Other attachments are also contemplated, such as an adhesive attachment.

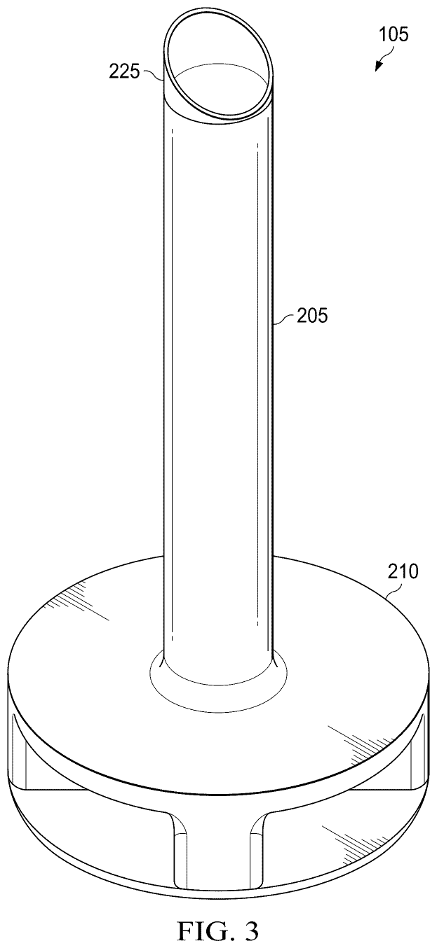

FIG. 3 is another perspective view of the cannula 105 of FIG. 2, illustrating additional details that may be associated with some embodiments. For example, the sleeve 205 may take a variety of forms that may be generally characterized as hollow prisms, and the sleeve 205 of FIG. 3 may be characterized geometrically as a hollow cylinder. In some embodiments, the sleeve 205 may be classified as a right circular hollow cylinder. The glare shield 225 may also take a variety of forms. In some embodiments, it may be advantageous for the glare shield 225 to take a form that is similar to the sleeve 205. For example, the glare shield 225 of FIG. 3 may also be characterized geometrically as a hollow cylinder. The glare shield 225 of FIG. 3 may be classified more specifically as a truncated right circular hollow cylinder, which may also be known as a cylindrical segment. In other variations, the glare shield 225 may be characterized as a half cylinder, half dome, scoop, or hood.

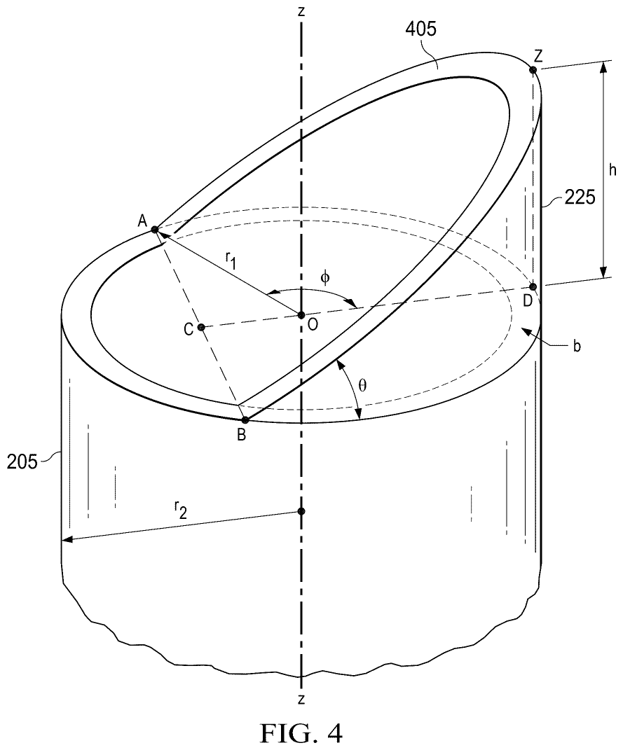

FIG. 4 is a detail view of FIG. 3, illustrating additional details that may be associated with some embodiments of the glare shield 225. For example, the glare shield 225 of FIG. 4 may be characterized geometrically, in part, by a base b having a center O. The base b may have a variety of shapes, including regular polygons or ellipses, or segments thereof. It may be advantageous in some embodiments for the base b to have an annular shape that can be readily joined to the sleeve 205.

The glare shield 225 of FIG. 4 comprises a face 405, which is generally annular and lies in a plane that is tilted at an angle θ to the base b which may have a face 415. The angle θ may vary generally in a range of greater than 0 degrees to less than 180 degrees, and preferably less than about 135 degrees. An angle θ of less than 90 degrees, as illustrated in the example of FIG. 4, may be advantageous for some embodiments. In the example of FIG. 4, the face 405 intersects the base b at a line AB having an endpoint A and an endpoint B, and the face 405 may be characterized as an elliptical segment. A line CD having an endpoint C and an endpoint D generally bisects line AB in the example of FIG. 4. In some embodiments, the base b may be further characterized by an arc AD that subtends an angle ϕ, which may vary generally in a range of greater than 0 degrees to less than or equal to 180 degrees. FIG. 4 illustrates an example in which the angle ϕ is greater than 90 degrees and less than 180 degrees. An arc AB may be symmetric about the line CD in some examples, so that the arc BD subtends an angle that is equal to the angle ϕ. In other examples, the arc BD may subtend an angle that is less than or greater than the angle ϕ.

The glare shield 225 of FIG. 4 may also be characterized, in part, by an apex Z, which is a point on the face 405 that coincides with the maximum height h of the face 405 from the base b, as measured normal to the base b. The glare shield 225 may be further characterized in some embodiments by an axis z that is normal to the base b and passes through the center O.

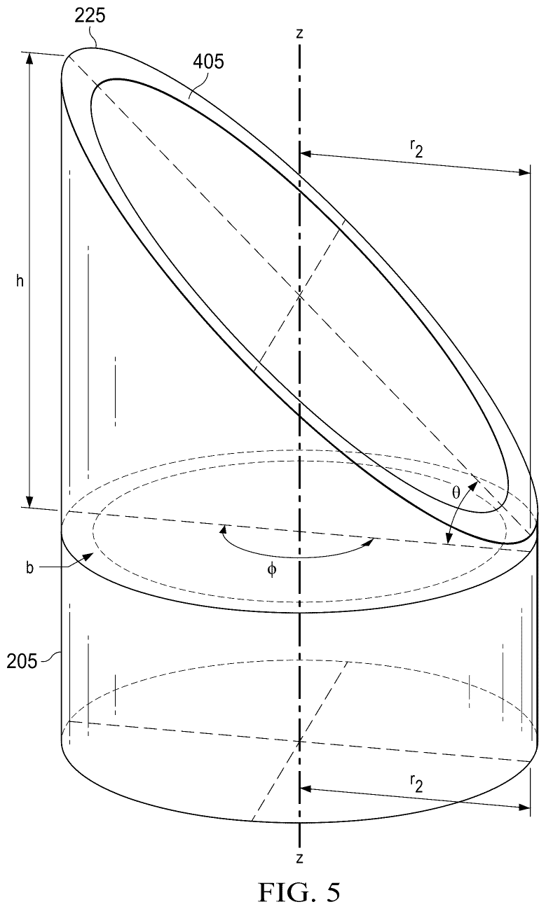

In the example of FIG. 4, the sleeve 205 and the glare shield 225 are coaxial about the axis z, and the sleeve 205 is coupled to the glare shield 225 at the base b. Additionally, the glare shield 225 of FIG. 4 has an outside radius r₁ that is equal to the outside radius r₂ of the sleeve 205, which provides a smooth surface transition between the sleeve 205 and the glare shield 225. In some embodiments, if the cannula 105 is used in ophthalmic surgery, the outside radius r₂ may be approximately 20, 23, 25, 27, or smaller gauge. In some embodiments, the maximum height h may be equal to the outside radius r₂ or some multiple or fraction thereof. For example, the height h may be approximately in a range of 1 to 10 times the outside radius r₂, such as in a range of 3 to 7 times the outside radius r₂, such as 5 times the outside radius r₂. As another example, the height h may be approximately in a range of ¼ to ¾ times the outside radius r₂, such as ½ times the outside radius r₂. Other values of height h are also contemplated. For example, in some embodiments, if the cannula 105 is used in ophthalmic surgery, height h may be approximately in a range of 0.25 millimeters to 5 millimeters, such as in a range of 1 to 3 mm, such as 2 millimeters. Again, other values of height h are also contemplated (such as 0.5 mm, 1 mm, 1.5 mm, 2 mm, 2.5 mm, 3 mm, etc.) Height h may be selected to be sufficient to block glare from an illuminator 110 disposed in the cannula. FIG. 5 is a perspective view of another example of the glare shield 225, illustrating additional details that may be associated with some embodiments. The example of FIG. 5 represents another embodiment of the general form of the glare shield 225 of FIG. 4, in which the face 405 intersects the base b at a single point. In this case, the endpoint A and the endpoint B are substantially coincident, and the angle ϕ is substantially equal to 180 degrees. The face 405 in the example of FIG. 5 may be characterized as an ellipse. In some embodiments, if the cannula 105 (shown in FIG. 5) is used in ophthalmic surgery, the outside radius r₂ may be approximately 20, 23, 25, 27, or smaller gauge. In some embodiments, the maximum height h (shown in FIG. 5) may be equal to the outside radius r₂ or some multiple or fraction thereof. For example, the height h may be approximately in a range of 1 to 10 times the outside radius r₂, such as in a range of 3 to 7 times the outside radius r₂, such as 5 times the outside radius r₂. As another example, the height h may be approximately in a range of ¼ to ¾ times the outside radius r₂, such as ½ times the outside radius r₂. Other values of height h are also contemplated. For example, in some embodiments, if the cannula 105 is used in ophthalmic surgery, height h may be approximately in a range of 0.25 millimeters to 5 millimeters, such as in a range of 1 to 3 mm, such as 2 millimeters. Again, other values of height h are also contemplated (such as 0.5 mm, 1 mm, 1.5 mm, 2 mm, 2.5 mm, 3 mm, etc.) Height h may be selected to be sufficient to block glare from an illuminator 110 disposed in the cannula.

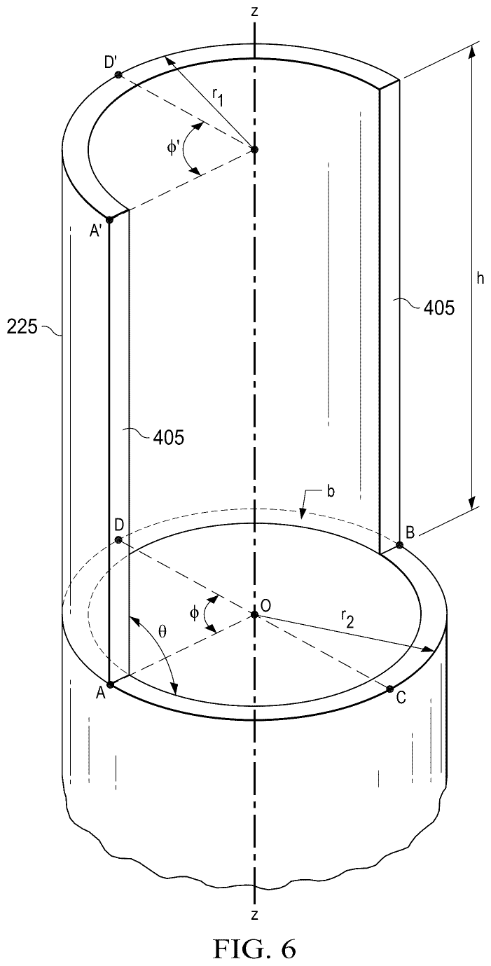

FIG. 6 is a perspective view of another example of the glare shield 225, illustrating additional details that may be associated with some embodiments. In the example of FIG. 6, the glare shield 225 may be characterized as a half cylinder, in which the angle ϕ is about 90 degrees and the angle θ is about 90 degrees. In other variations, the angle θ may be less than 90 degrees. The angle ϕ may vary generally in a range of greater than or equal to 0 degrees to less than or equal to 180 degrees. In some embodiments, the glare shield 225 may have a distal end that may be characterized by an arc A′D′ that subtends an angle ϕ′ which may vary generally in a range of greater than or equal to 0 degrees to less than or equal to 180 degrees. In the example of FIG. 6, the angle ϕ is substantially equal to the angle ϕ′ In other examples, the angle ϕ′ may be less than the angle ϕ. For example, in some embodiments, angle ϕ′ may be approximately in a range of 0 to 20 degrees smaller than angle ϕ, such as in a range of 5 to 15 degrees smaller than angle ϕ, such as 10 degrees smaller than angle ϕ. In other embodiments, angle ϕ′ may be approximately ¼ to ¾ times angle ϕ, such as approximately ½ times angle ϕ. In some embodiments, if the cannula 105 (shown in FIG. 6) is used in ophthalmic surgery, the outside radius r₂ may be approximately 20, 23, 25, 27, or smaller gauge. In some embodiments, the maximum height h (shown in FIG. 6) may be equal to the outside radius r₂ or some multiple or fraction thereof. For example, the height h may be approximately in a range of 1 to 10 times the outside radius r₂, such as in a range of 3 to 7 times the outside radius r₂, such as 5 times the outside radius r₂. As another example, the height h may be approximately in a range of ¼ to ¾ times the outside radius r₂, such as ½ times the outside radius r₂. Other values of height h are also contemplated. For example, in some embodiments, if the cannula 105 is used in ophthalmic surgery, height h may be approximately in a range of 0.25 millimeters to 5 millimeters, such as in a range of 1 to 3 mm, such as 2 millimeters. Again, other values of height h are also contemplated (such as 0.5 mm, 1 mm, 1.5 mm, 2 mm, 2.5 mm, 3 mm, etc.) Height h may be selected to be sufficient to block glare from an illuminator 110 disposed in the cannula.

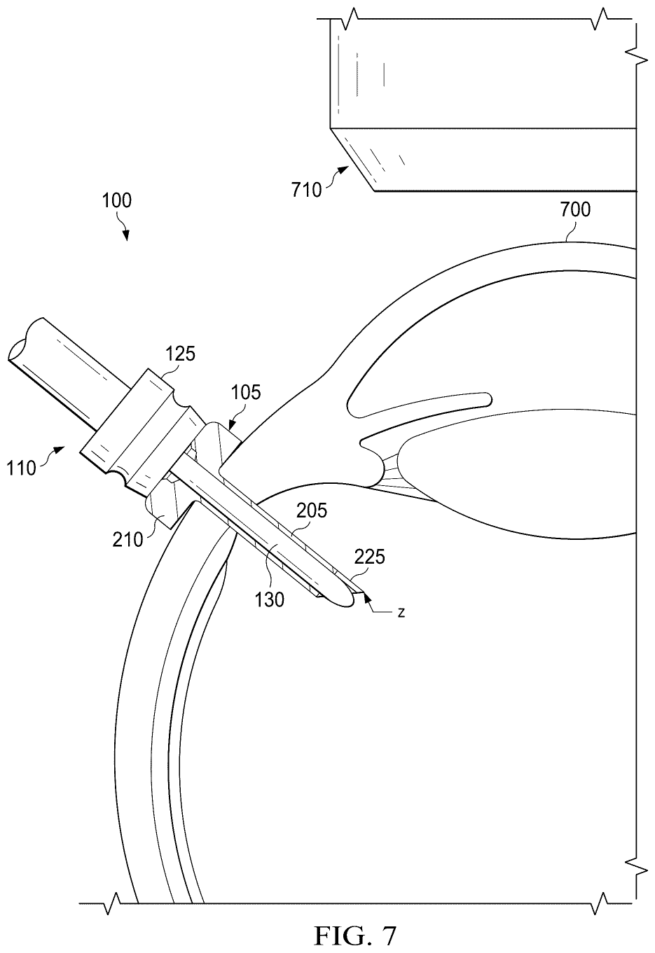

FIG. 7 is a schematic diagram of a method for using the system 100 with an eye 700. Generally, a trocar (not shown) can be used with the cannula 105 to make an incision into the eye 700, allowing the cannula 105 to be inserted into the eye 700 until the hub 210 contacts the eye 700. The trocar can be removed while leaving the cannula 105 in place. The illuminator 110 of FIG. 7 can be inserted through the hub 210 and the sleeve 205. In some examples, the stem 130 can extend past the sleeve 205 until aligned with the apex Z of the glare shield 225.

In operation, the light source 115 can transmit incoherent light through the illuminator 110 via cable 120 to provide diffuse illumination (also referred to as chandelier illumination) over a wide area of the interior of the eye 700, which can be viewed through a surgical microscope 710. The glare shield 225 can at least partially block light emitted from the illuminator 110, which can substantially reduce or prevent glare from the end of the stem 130 back through the visual axis of the surgical microscope 710. In some examples, the glare shield 225 may be rotated around the illuminator 110 to control the illumination. For example, the glare shield 225 of FIG. 7 may be rotated around the stem 130 to control illumination.

The systems, apparatuses, and methods described herein may provide significant advantages. For example, while chandelier lighting systems can provide a stationary and diffuse form of endoillumination from an anterior position, such lighting may produce a significant amount of glare, particularly under air. Some features of the system 100, such as the glare shield 225, may substantially reduce or eliminate such glare, without adding complexity to the system 100 or requiring additional steps or incisions for surgical procedures.

While shown in a few illustrative embodiments, a person having ordinary skill in the art will recognize that the systems, apparatuses, and methods described herein are susceptible to various changes and modifications that fall within the scope of the appended claims.

Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations, the light source 115 and the cannula 105 may be combined or sold separately.

The claims may also encompass additional subject matter not specifically recited in detail. Features, elements, and aspects described in the context of some embodiments may also be omitted, combined, or replaced by alternative features serving the same, equivalent, or similar purpose without departing from the scope of the appended claims.

What is claimed is:

1. A system for surgery on an eye, the system comprising:
a cannula having a hub with an instrument port, a shield, and a sleeve having a first end coupled to the instrument port and a second end coupled to the shield; and
an illuminator having a first end configured to be coupled to a light source and a second end configured to be inserted through the instrument port and the sleeve;
wherein the shield is configured to partially block light emitted from the second end of the illuminator.

2. The system of claim 1, wherein the shield comprises a truncated hollow cylinder.

3. The system of claim 1, wherein the shield comprises a half cylinder.

4. The system of claim 1, wherein the shield and the sleeve are coaxial.

5. The system of claim 1, wherein:
the shield and the sleeve are coaxial;
the shield has a first outside radius; and
the sleeve has a second outside radius equal to the first outside radius.

6. The system of claim 1, wherein the shield comprises:
a base coupled to the sleeve; and
a face that is tilted at an angle to the base.

7. The system of claim 6, wherein the angle is less than or equal to 90 degrees.

8. The system of claim 6, wherein:
the face intersects the base; and
the angle is less than or equal to 90 degrees.

9. The system of claim 1, wherein:
the shield comprises a base coupled to the sleeve, and a face that is tilted at an angle θ to the base;
the base is characterized by a center and an arc that subtends an angle ϕ having a vertex at the center;
the angle θ is less than or equal to 90 degrees; and
the angle ϕ is less than or equal to 180 degrees.

10. The system of claim 1, wherein the shield is configured to be rotated around the illuminator.

11. A system for surgery on an eye, the system comprising:
a light source configured to provide an incoherent light;
a cannula having a hub with an instrument port, a shield, and a sleeve having a first end coupled to the instrument port and a second end coupled to the shield; and
an illuminator having a first end configured to be coupled to the light source and a second end configured to be inserted through the instrument port and the sleeve;
wherein the sleeve and the shield are configured to be inserted into the eye, and the shield is configured to partially block light emitted from the second end of the illuminator in the eye.

12. The system of claim 11, wherein:
the shield and the sleeve are coaxial;
the shield has a first outside radius; and
the sleeve has a second outside radius equal to the first outside radius.

13. The system of claim 11, wherein:
the shield comprises a base coupled to the sleeve, and a face that is tilted at an angle θ to the base;
the base is characterized by a center and an arc that subtends an angle ϕ having a vertex at the center;
the angle θ is less than or equal to 90 degrees;
the angle ϕ is less than or equal to 180 degrees; and
the face intersects the base.

14. The system of claim 13, wherein the shield is configured to be rotated around the illuminator.

15. An apparatus for eye surgery, the apparatus comprising:
a light source configured to provide an incoherent light;
a cannula having a hub with an instrument port, a sleeve, and a shield; and
an illuminator configured to be coupled to the light source;
wherein the sleeve and the shield are coaxial;
wherein the shield has a first outside radius, and the sleeve has a second outside radius equal to the first outside radius;
wherein the shield comprises a base coupled to the sleeve, and a face that intersects the base at an angle θ that is less than or equal to 90 degrees;
wherein the base is characterized by a center, and an arc that subtends an angle ϕ that is less than or equal to 180 degrees and has a vertex at the center; and
wherein the sleeve and the shield are configured to be inserted into an eye, the illuminator is configured to be inserted through the instrument port and the sleeve, the shield is configured to be rotated around the illuminator and to partially block light emitted from the illuminator into the eye.
